(19) United States
(12) Patent Application Publication
(10) Pub. No.: US 2017/0290632 A1
(43) Pub. Date: Oct. 12, 2017

(54) MEDICAL ROBOT COVER

(71) Applicant: DENSO CORPORATION, Kariya-city, Aichi-pref. (JP)

(72) Inventors: Ayako NAKATSU, Minato-ku, Tokyo (JP); Hiroyuki HOJO, Minato-ku, Tokyo (JP); Jiro KATO, Minato-ku, Tokyo (JP); Hideki OKUDA, Kariya-city (JP); Satoru NAKAMURA, Kariya-city (JP); Minoru TAKAHASHI, Kariya-city (JP)

(21) Appl. No.: 15/509,712

(22) PCT Filed: Sep. 8, 2015
(86) PCT No.: PCT/JP2015/004568

§ 371 Date: Mar. 8, 2017

(30) Foreign Application Priority Data
Sep. 22, 2014 (JP) 2014-192992

(51) Int. Cl. A61B 46/10; A61B 34/35; B25J 19/00

(52) U.S. Cl. CPC A61B 46/10; B25J 19/0075; A61B 34/35

(57) ABSTRACT

A medical robot cover covers an external surface of a medical robot including: a main unit that stands on a base; and an arm member attached to one end of the main unit in a swingable or extensible manner. The medical robot cover includes: an attaching portion to be wound around at least one of the main unit and the arm member, on an inner circumferential surface of the cover facing the external surface of the medical robot. Therefore, the medical robot cover can be easily placed on the medical robot. Further, the medical robot cover can be restricted from being damaged due to extension or swinging of the arm member, and can be restricted from inhibiting the action of the arm member.

### CROSS REFERENCE TO RELATED APPLICATION

This application is based on Japanese Patent Application No. 2014-192992 filed on Sep. 22, 2014, the disclosure of which is incorporated herein by reference.

### TECHNICAL FIELD

The present disclosure relates to a medical robot cover for a robot conducting a surgical operation.

### BACKGROUND ART

Generally in conducting a surgical operation, medical devices to be used in the surgical operation are placed in a clean field by covering the medical devices that are considered as unclean with a sterilized cover or the like to prevent entry of bacteria or the like from an incisional wound or the like of the patient. That is, by covering medical devices with a sterilized cover or the like, an aseptic condition is provided.

In recent years, operation procedures in which a surgeon performs an operation by manipulating a robot or the like, such as a robot-assisted surgical operation or a telerobot surgical operation are conducted. A medical robot for use in such a robot-assisted surgical operation, a telerobot surgical operation or the like should be clean for the same reason as described above.

A variety of forms are known as such a sterilizing method, and for example, a sterilized cover described in Patent Literature 1 covers an unsterilized part of a robot system for surgical operation. The sterilized cover has an integrated cuff and a fastener. The integrated cuff includes an outer surface neighboring a sterile field for performing a surgical operation, an inner surface forming a cavity that receives the unsterilized part of the robot system for surgical operation, a permanently folded back fold of the outer surface and the inner surface in an open end of the cavity, and markers that is provided on the permanently folded fold and that specify a sterile side and a nonsterile side of the drape. The fastener is bound to the outer surface, and fastens the sterilized cover to the unsterilized part of the robot system for surgical operation while reducing the capacity of the sterilized cover.

Such a sterilized cover protects the system and the operating patient, and exerts improved efficiency and efficacy of the robot surgical operation by being designed so that it is easily installed, and the installation time is minimized while the maximum degree of freedom of operation and visibility during a surgical operation are ensured.

### PRIOR ART LITERATURES

### Patent Literature

Patent Literature 1: JP 2004-238773 A

### SUMMARY OF INVENTION

As a result of detailed examinations, the inventors found the issue that when a conventional sterilized cover is used for a medical robot having a main unit and an arm member that largely extends/contracts or swings with respect to the main unit, the sterilized cover is damaged by being caught between the arm member and a drive part of the main unit, or the sterilized cover restricts the motion of the robot, or the sterilized cover hinders the manipulation, in association with extension/contraction or swing of the arm member. The degree of freedom in extending/contracting or swinging operation of the arm member is not sufficient, and the issue of hindering such an operation was found.

In recent surgical operations, operations using an endoscopic operation robot that are minimally invasive to patients are conducted. Since such an endoscopic operation robot is accompanied by a small wound and less postoperative pain, the operative procedure allows early rehabilitation and is expected as a widely prevailing operative procedure.

Even when such an endoscopic operation robot is used, it is necessary to place the endoscopic operation robot in a clean field by using a sterilized cover likewise medical robots used in a robot-assisted surgical operation, a telerobot surgical operation or the like as described above, and a sterilized cover that will not hinder the motion of the arm member is demanded so as to reliably transmit a small motion to the arm member.

It is an object of the present disclosure to provide a medical robot cover for use in a medical robot having a main unit, an arm member attached to the main unit in an extendable/contractible or swingable manner, featured in that easy installation is enabled, the medical robot cover is not damaged by extension/contraction or swing of the arm member, and motion of the arm member is not hindered, and manipulation is not hindered.

According to an aspect of the present disclosure, a medical robot cover covers an external surface of a medical robot including: a main unit that stands on a base; and an arm member attached to one end of the main unit in a swingable or extensible manner. The medical robot cover includes: an attaching portion to be wound around at least one of the main unit and the arm member, on an inner circumferential surface of the cover facing the external surface of the medical robot.

The medical robot cover may further include: a body part that covers the main unit; and an arm part that covers the arm member. The attaching portion may include: a first attaching portion attached to an inner circumferential surface of the body part facing the main unit; and a second attaching portion to be wound around the arm part together with the arm member on the external surface of the arm part. The body part may have a motion space allowing extension/contraction or swing of the arm member. The arm part may have a fastening portion that fastens the arm part to the arm member.

With this configuration, since the inner circumferential surface of the cover facing the external surface of the medical robot is provided with an attaching portion that is wound at least one of the main unit and the arm member, it is possible to install the medical robot cover in the medical robot in such a manner that the motion of the arm member is not hindered even when the arm member extend/contracts or swings with respect to the main unit.

Since the body part that covers the main unit, and the arm part that covers the arm member are provided, it is possible to reliably handle the medical robot aseptically, and to prevent damage of the medical robot cover in association with the motion of the arm member without hindering the motion.

Since the first attaching portion attached to the inner circumferential surface facing the main unit, in the body part, and the second attaching portion to be wound around the arm part together with the arm member, on the external surface of the arm part are included, it is possible to allow extension/contraction, swing or the like operation of the arm member by connecting the main unit and the inner circumferential surface of the body part by the first attaching portion, and it is possible to prevent the medical robot cover from coming into contact with an operative field by preventing the arm part from being slack by winding the second attaching portion around the external surface of the arm part.

Since the body part has the motion space for allowing extension/contraction or swing of the arm member, the operation such as extension/contraction or swing of the arm member will not be hindered even when the medical robot cover is attached to the medical robot.

Since the fastening portion that fastens the arm part to the arm member is provided, even when the arm member extends/contracts or swings, it is possible to prevent the arm part from slipping with respect to the arm member, and to prevent the arm part from being slack, and to prevent occurrence of a wrinkle.

### BRIEF DESCRIPTION OF DRAWINGS

The above and other objects, features and advantages of the present disclosure will become more apparent from the following detailed description made with reference to the accompanying drawings.

FIG. 1 is a view illustrating a medical robot cover according to an embodiment, which is attached to a medical robot.

FIG. 2 is a configuration view illustrating the medical robot cover of the embodiment.

FIG. 3 is a folded view of the medical robot cover of the embodiment.

FIG. 4 is a view illustrating a procedure attaching the medical robot cover of the embodiment.

FIG. 5 is a view illustrating a procedure attaching the medical robot cover of the embodiment.

FIG. 6 is a view illustrating a procedure attaching the medical robot cover of the embodiment.

### DESCRIPTION OF EMBODIMENTS

Hereinafter, a medical robot cover will be described by referring to drawings. The following embodiment does not limit each claim, and not every combination of features described in the embodiment is necessary.

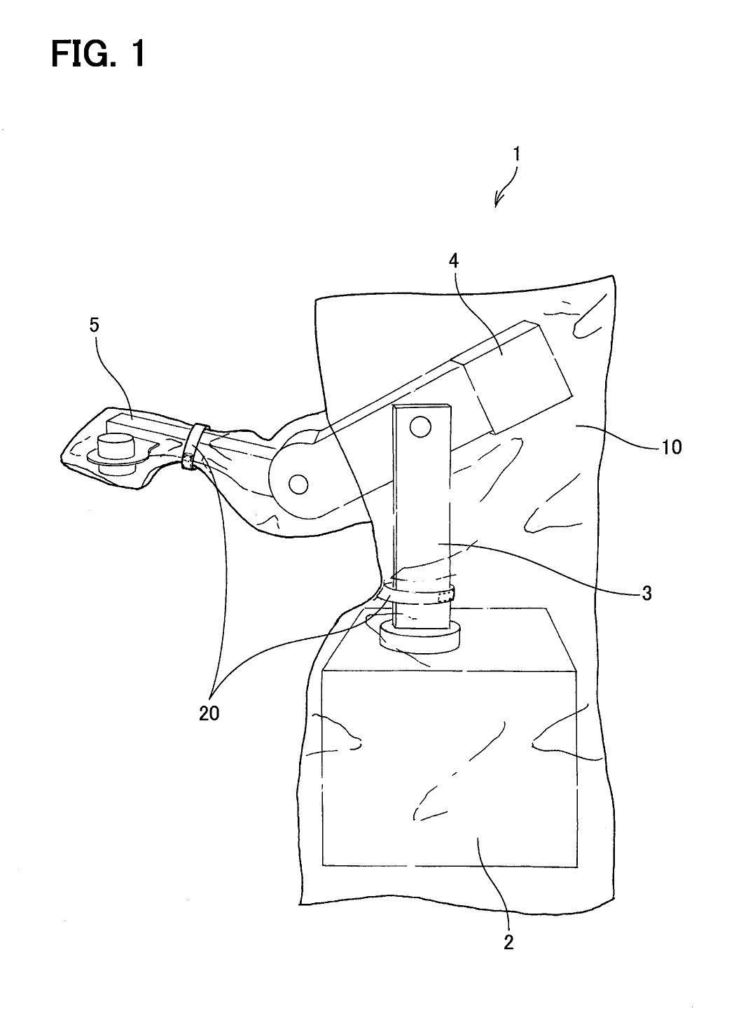

As shown in FIG. 1, a medical robot cover 10 according to the present embodiment is attached to a medical robot 1 having a main unit 3 standing on a base 2, and an arm member 4 attached to one end of the main unit 3 in a swingable and extensible manner, and the medical robot cover 10 conducts sterilization during an operation.

In the medical robot 1, the arm member 4 that is manipulated in a swingable and extensible manner is attached to the main unit 3 so that a physician can perform an operation while viewing an image of an endoscope (not shown) and the motion of the hand of the physician can be accurately reproduced. To a distal end portion 5 of the arm member 4, a surgical instrument such as forceps or an electric knife is attached. Thus the medical robot 1 is configured to enable an accurate operation to be conducted more simply by having a range of motion larger than that of a human wrist while accurately realizing the motion of the hand of the physician.

As described above, since the medical robot 1 enables an operation with such a small wound through which an endoscope, forceps, and the like can insert without performing a laparotomy, it is possible to perform a diagnosis and a treatment with small invasion into the body of the patient.

The forceps or the like attached to the distal end portion 5 can be driven by the control of a pushing or drawing operation of wire or the like, or by the use of pneumatic pressure. In any case regardless of the employed driving method, an intuitive operation is enabled, and the burden on the physician during the operation can be reduced with such a configuration that a touch at the distal end of the forceps can be fed back to the physician.

Since the inside of an operating room is a sterilized, so-called, clean field, it is necessary to perform an operation while covering the external surface of the medical robot 1 with the medical robot cover 10 so as to define a boundary with an unsterilized so-called unclean field in using the medical robot 1.

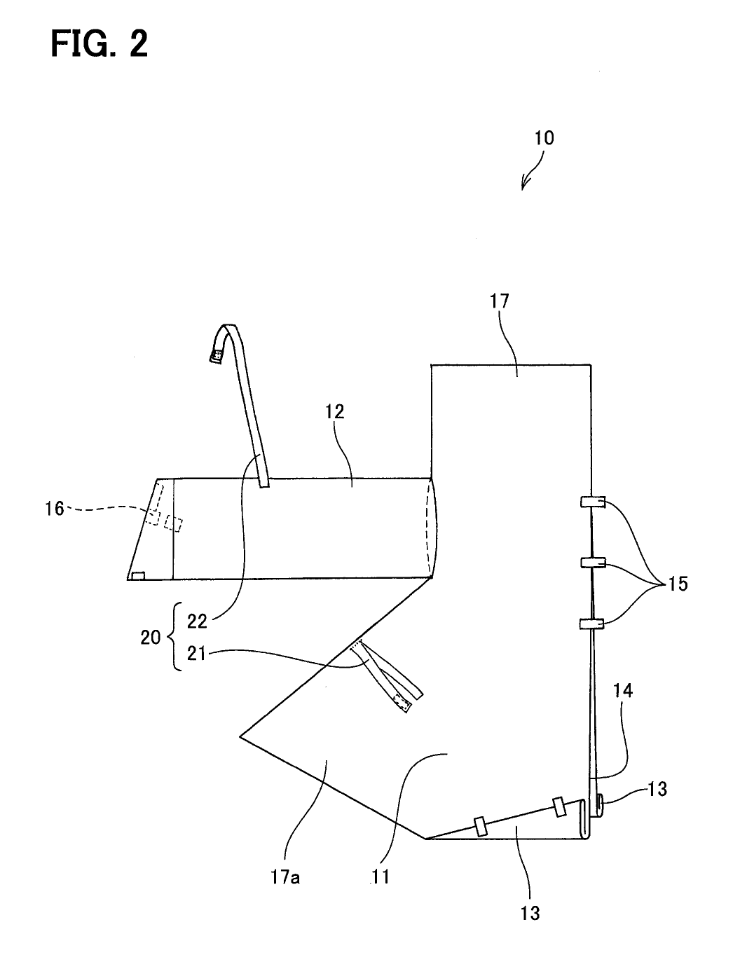

As shown in FIGS. 1 and 2, the medical robot cover 10 according to the present embodiment has a body part 11 that covers the external surfaces of the main unit 3 and the base 2, and an arm part 12 that covers the external surface of the arm member 4. On the main unit 3 and the arm member 4, an attaching portion 20 including a first attaching portion 21 and a second attaching portion 22, is wound around, and attachment and fixation to the medical robot 1 is achieved by the attaching portion 20.

As shown in FIG. 2, the body part 11 of the medical robot cover 10 according to the present embodiment is formed into a bag shape having a closed top side and an open bottom side. To one end of the body part 11 in the transverse direction, the arm part 12 formed into a bag shape is attached, and the other end to which the arm part 12 is attached is not closed together, and an opening 14 is formed. The size of the opening of the opening 14 can be adjusted by a fastening part 15 formed by a hook-and-loop fastener or an adhesive tape, and after attachment to medical robot 1, the opening 14 can be closed by the fastening part 15. The arm part 12 is attached to one end of the body part 11 in the transverse direction at a position offset from the top side of the body part 11, and thus a motion space 17 is formed in an upper part of the body part 11.

The edge of the body part 11 on the side to which the arm part 12 is attached is formed into a substantially triangular shape, and this triangular shape forms a second motion space 17a. By means of the second motion space 17a, the bottom end of the body part 11 is prevented from reaching the floor, and a motion space, for example, for rotation of the arm member 4 is formed. The motion space 17 and the second motion space 17a can be formed depending on the motion of the medical robot 1, extension/contraction or swing of the arm member 4, rotation of the main unit and so on, and only one of the motion space 17 and the second motion space 17a may be provided as necessary.

Since the medical robot cover 10 according to the present embodiment has the opening 14 and the fastening part 15 in the body part 11, it is possible to reliably provide a clean field and to give a large opening when it is attached to the medical robot 1, and hence it becomes possible to conduct the attaching operation more easily.

The bottom side of the body part 11 is formed with a rollup part 13 in which an end part of the body part 11 is rolled up. The rollup part 13 is so configured that when the length of the body part 11 is short, sterilization is ensured up to the bottom end of the medical robot 1, by adjusting the length of the body part 11 reliably depending on the height of the medical robot 1 to which the medical robot cover 10 according to the present embodiment is attached. When the length of the body part 11 is long, it is possible to appropriately adjust the rolling-up length so as to prevent the lower end of the medical robot cover 10 from coming into contact with the floor to become unclean, and to prevent the operator from stepping on the lower end, and to prevent the lower end from being rolled-in under the medical robot 1.

To the inner circumferential surface of the body part 11, the first attaching portion 21 is attached. The first attaching portion 21 is a belt-shaped member, and the belt-shaped member is folded in substantially half, and the folded point is attached to the inner circumferential surface of the body part 11 by heat welding, adhesion and the like means. Thus, the first attaching portion 21 is formed in such a manner that both end parts are free ends with respect to the inner circumferential surface of the body part 11. A uniting part for closing the both end parts is attached to the both end parts of the first attaching portion 21. Specifically, as the uniting part, a hook-and-loop fastener, a double-sided adhesive tape, or the like is preferably used.

On the outer circumferential surface of the arm part 12, the second attaching portion 22 is attached. Unlike the first attaching portion 21, the second attaching portion 22 is so configured that one end is attached to the outer circumferential surface of the arm part 12 and the other end is formed as a free end, and to the end part formed as a free end, a uniting part is attached as is the case with the first attaching portion 21. Alternatively, part of the rubber formed into a ring may be attached to the outer circumferential surface of the arm part 12, and the distal end of the arm member 4 may be penetrated in the ring-like rubber. In the manner as described above, in the medical robot cover 10 according to the present embodiment, an attaching portion is formed by the first attaching portion 21 and the second attaching portion 22.

Further, in a distal end of the arm part 12 (left side in FIG. 2), an fastening portion 16 is attached to the inner circumferential surface. Although the fastening portion 16 may have any configuration as long as the arm part 12 can cover the distal end portion 5 of the arm member 4 without slipping off from the distal end portion 5 of the arm member 4, for example, a double-sided adhesive tape is preferably used. The fastening portion 16 provides a mark of the starting point in covering the medical robot 1 with the medical robot cover 10, and has the effect of facilitating the covering operation. Therefore, the fastening portion 16 preferably has a shape indicating a starting point, specifically, a round or square that is a dot, or a triangle or an arrow and so on. When the fastening portion 16 is not intended to fasten to the medical robot 1, or intended to have a configuration of only a mark, a mark may be added only at the distal end of the arm part 12 rather than the fastening portion 16.

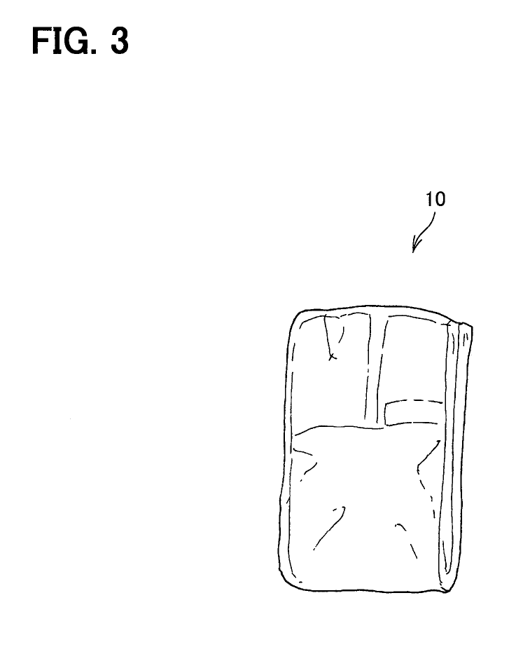

Next, referring to FIGS. 3 to 6, an attaching procedure of the medical robot cover 10 according to the present embodiment will be described.

The medical robot cover 10 according to the present embodiment is folded down as shown in FIG. 3 in the initial condition. The medical robot cover 10 according to the present embodiment is folded down inside out so that the inner circumferential surface of the medical robot cover 10 is outside for conducting a clean operation because the inner circumferential surface is to come into contact with the external surface of the medical robot 1 which is an unclean field, and the outer circumferential surface of the medical robot cover 10 should be a clean field. Specifically, since the medical robot cover 10 is folded down inside out so that the inner circumferential surface is outside, an operator in an unclean field can perform the attachment operation of the medical robot cover 10 without necessity of washing his/her hands.

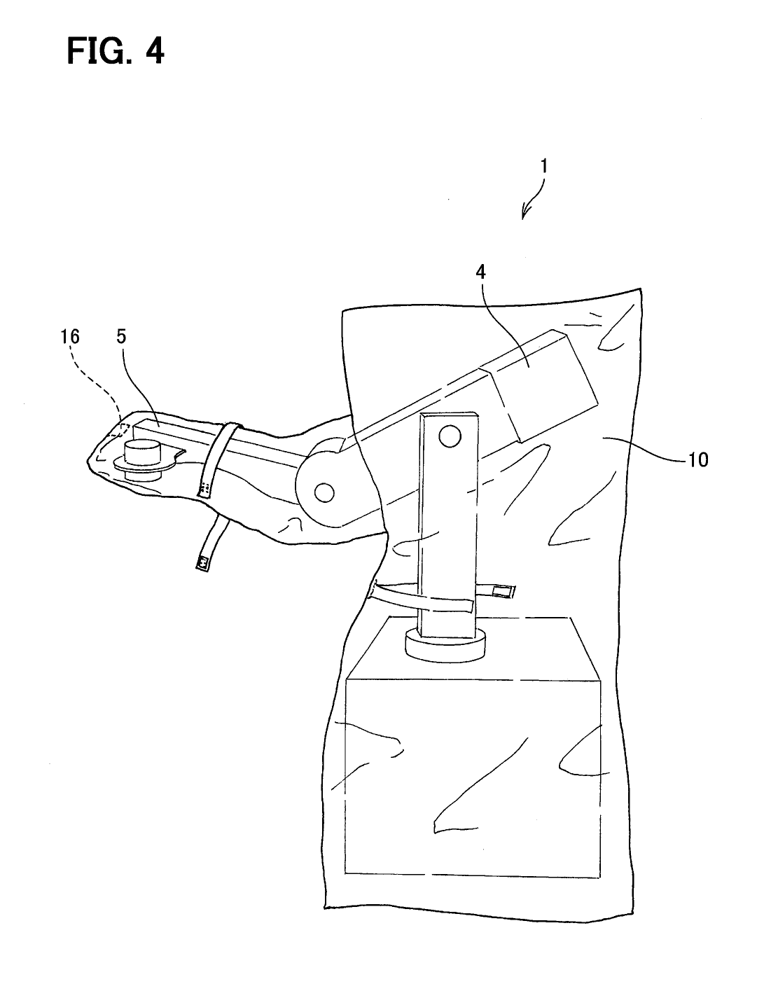

As shown in FIG. 4, the paper liner of the double-sided adhesive tape 16 as the fastening portion is removed, and the distal end of the arm part 12 adheres and is fixed to the distal end portion 5 of the arm member 4. Then, the arm member 4 is covered with the arm part 12. The opening 14 is opened, and attachment is conducted so that the main unit 3 and the base 2 are covered with the body part 11.

Since the distal end portion 5 adheres and is fixed to the distal end of the arm part 12 in the manner as described above, a wrinkle occurring otherwise in the arm part 12 will not hinder the arm member 4 or the operation of the surgical instrument attached to the distal end portion 5 of the arm member 4 when the arm member 4 is moved.

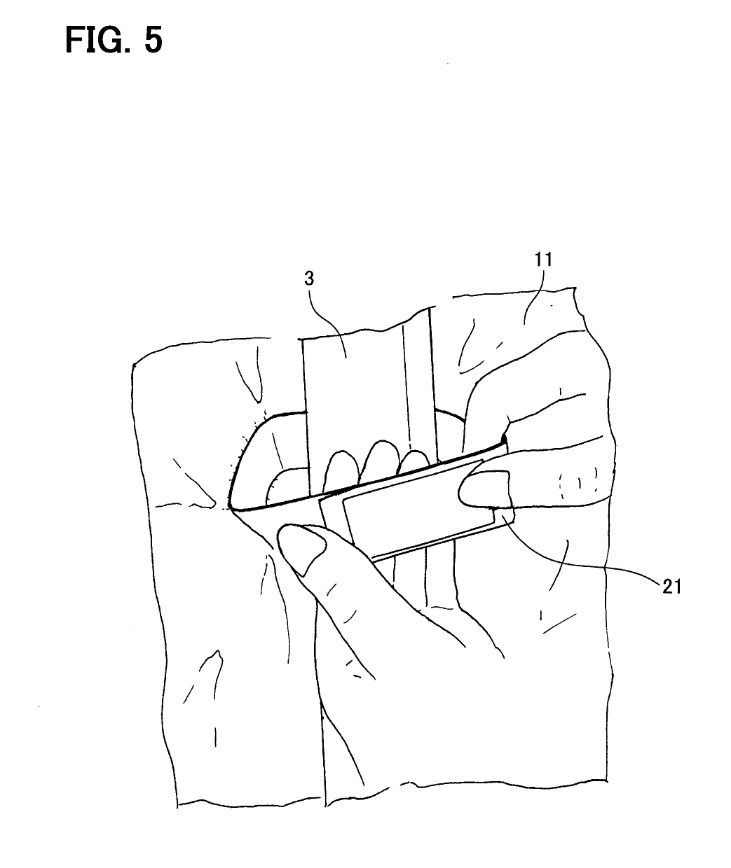

Then as shown in FIG. 5, the first attaching portion 21 is wound around the main unit 3 in such a manner that the both ends thereof unite. At this time, it is preferred to paste the first attaching portion 21 to such a degree that the both ends lightly unite. By attaching in this manner, the degree of freedom of the body part 11 with respect to the main unit 3 increases, and attachment can be made so that when the arm member 4 swings or extends/contracts, the medical robot cover 10 according to the present embodiment will not hinder the operation. In contrast to this, when the first attaching portion 21 is attached from the outer circumferential surface, for example, swinging of the arm member 4 is hindered because the first attaching portion 21 does not turn in the horizontal direction. Since the first attaching portion 21 is present on the inner circumferential surface, an attachment operation can be made by a circulating nurse. As described above, the first attaching portion 21 is preferably attached to the inner circumferential surface because the first attaching portion 21 is the site that comes into contact with an unclean medical device.

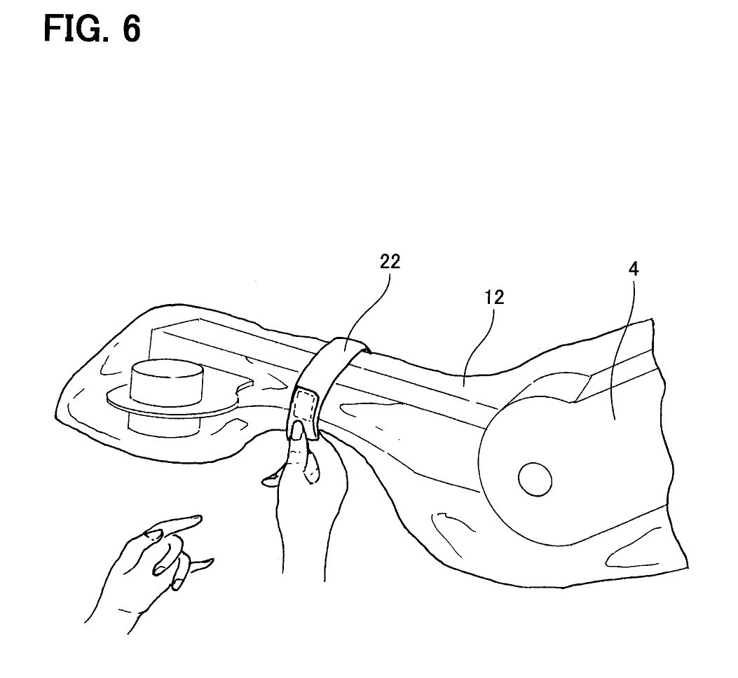

Next, as shown in FIG. 6, the second attaching portion 22 attached to the outer circumferential surface of the arm part 12 is wound around the arm part 12 together with the arm member 4 to attach the arm part 12 to the arm member 4. In this way, since the outer circumferential surface of the arm part 12 can be attached by the second attaching portion 22 so that the arm part 12 is not slack, it becomes possible to achieve attachment in such a manner that even when the arm member 4 conducts extending/contracting or swinging operation, the operation is not hindered. Since the site where the second attaching portion 22 is attached is adjacent to an operative field, it is possible to prevent the cover from bending and hindering the manipulation. This operation is required to be performed by an operator in a clean field such as an operating surgeon or a scrub nurse who has washed his/her hands because this operation involves touching the outer circumferential surface of the medical robot cover 10 according to the present embodiment. However, since there is a case that an operating surgeon or an assistant needs to perform this operation for the reason as described above, the second attaching portion 22 is preferably situated on the outer circumferential surface.

The opening 14 is closed by the fastening part 15 after the medical robot cover 10 according to the present embodiment is attached to the medical robot 1. Regarding the fastening part 15, while the configuration of fixing at three points is illustrated in FIG. 2, the number of fastening part may be increased or decreased as necessary, and a configuration that allows continuous fastening over the required length in the vertical direction of FIG. 2 may be employed. Since the fastening part 15 is preferably fastened by an engaged person who touches the unclean field such as a circulating nurse, it is preferably attached to the inner circumferential surface of the medical robot cover 10.

As described above, since the medical robot cover 10 according to the present embodiment has the second attaching portion 22 that adheres to and fixes the distal end portion 5 of the arm member 4, and winds the external surface of the arm part 12 together with the arm member 4, it is possible to prevent occurrence of an unneeded slack or wrinkle in the arm part 12, and thus operation such as extension/contraction or swing of the arm member 4 will not be hindered.

In contrast to this, the body part 11 is fixed to the main unit 3 by the first attaching portion 21 attached to the inner circumferential surface facing the main unit 3. Therefore, even when the arm part 12 is drawn in association with the extension/contraction and swing of the arm member 4, the body part 11 appropriately moves in association with the movement of the arm part 12, so that the operation such as extension/contraction or swing of the arm member 4 will not be hindered. Further, since the motion space 17 is formed in an upper part of the body part 11, the arm member 4 can move inside the motion space 17 freely. This prevents the body part 11 from being drawn undesirably in association with extension/contraction or swing of the arm member 4.

While the embodiment has been described in the above, the technical scope of the present disclosure is not limited to the description of the above embodiment. The above embodiment may be changed or modified in various ways.

For example, in the above embodiment, the description has been made for the case where the medical robot 1 used for an endoscopic operation is made clean by attaching a surgical instrument to the distal end portion 5 of the arm member 4, however, the objective medical robot for which provision of a clean field is required is not limited to this, and may be, for example, a hand base robot in which the distal end portion 5 is provided with a hand base for placement of a hand of a physician, and the surgical operability is improved by reducing the trembling of the arm and fatigue of the physician in such a manner that the base stands still to support the arm of the physician during a surgical operation, and moves to follow the arm of the physician when the physician changes the position of his/her arm.

The description has been made for the case where the first and the second attaching portions 21, 22 are attached to the body part 11 and the arm part 12, respectively, the number of each of the first and the second attaching portions 21, 22 is not limited and can be increased or decreased appropriately as necessary.

Such changes and modifications are to be understood as being within the scope of the present disclosure as defined by the appended claims.

What is claimed is:

1. A medical robot cover that covers an external surface of a medical robot, the medical robot including: a main unit that stands on a base; and an arm member attached to one end of the main unit in a swingable or extensible manner, the medical robot cover comprising:
an attaching portion to be wound around at least one of the main unit and the arm member, on an inner circumferential surface of the cover facing the external surface of the medical robot.

2. The medical robot cover according to claim 1, further comprising:
a body part that covers the main unit; and
an arm part that covers the arm member.

3. The medical robot cover according to claim 2, wherein
the attaching portion includes:
a first attaching portion attached to an inner circumferential surface of the body part facing the main unit; and
a second attaching portion to be wound around the arm part together with the arm member on the external surface of the arm part.

4. The medical robot cover according to claim 2, wherein
the body part has a motion space allowing extension/contraction or swing of the arm member.

5. The medical robot cover according to claim 2, wherein
the arm part has a fastening portion that fastens the arm part to the arm member.
